(19) United States
(12) Patent Application Publication
(10) Pub. No.: US 2023/0029768 A1
(43) Pub. Date: Feb. 2, 2023

(54) PROCESS FOR PRODUCING CYCLOHEXANOL AND CYCLOHEXANONE

(71) Applicant: BASF FRANCE SAS, Levallois-Perret (FR)

(72) Inventors: Maxime HUCHEDE, Lyon (FR); Thi Thu Huong PHAN, Saint Symphorien d'Ozon (FR)

(21) Appl. No.: 17/785,092

(22) PCT Filed: Dec. 17, 2020
(86) PCT No.: PCT/EP2020/086718

§ 371 Date: Jun. 14, 2022

(30) Foreign Application Priority Data
Dec. 18, 2019 (EP) 19217305.2

(51) Int. Cl. C07C 29/17; C07C 49/403; C07C 45/53; B01J 25/02; C07C 35/08

(52) U.S. Cl. CPC C07C 29/172; C07C 49/403; C07C 45/53; B01J 25/02; C07C 35/08

(57) ABSTRACT

The invention concerns a method for preparing a mixture containing cyclohexanol and cyclohexanone, comprising the step of hydrogenating cyclohexyl hydroperoxide in cyclohexane in the presence of a Raney nickel catalyst to give cyclohexanol and cyclohexanone.

### BACKGROUND ART

Several different processes have been used for the oxidation of cyclohexane into a product mixture containing cyclohexanone and cyclohexanol. Such product mixture is commonly referred to as a KA oil (ketone/alcohol oil) mixture. The great majority of KA oil is consumed in the production of precursors to Nylon 6,6 and Nylon 6. The KA oil mixture can be readily oxidized to produce adipic acid, which is an important reactant in processes for preparing certain condensation polymers, notably polyamides, in particular Nylon 6,6. Given the large quantities of adipic acid consumed in these and other processes, there is a need for cost-effective processes for producing adipic acid and its precursors. Furthermore, cyclohexanol from KA oil can be dehydrogenated to give cyclohexanone, and cyclohexanone from KA oil and the dehydrogenation of cyclohexanol can be reacted, preferably with hydroxylamine via cyclohexanonoxim, to give epsilon-caprolactame.

Classical process to produce a mixture containing cyclohexanone and cyclohexanol is conducted in two steps to get KA oil through oxidation of cyclohexane. First, the thermal auto-oxidation of cyclohexane leads to the formation of cyclohexyl hydroperoxide (CyOOH) that is isolated. The second step, KA oil is obtained through the decomposition of CyOOH which is catalyzed by using chromium ions or cobalt ions as homogenous catalysts.

With the regulation restrictions all over the world, the requirement of replacement of environmentally unfriendly catalysts, such as chromium and cobalt catalysts, becomes more and more urgent. The environmental footprint and the economics of this process could be significantly improved if the current homogeneous catalysts could be replaced by non-toxic heterogeneous catalysts.

Various types of homogeneous catalysts have been used to catalyze oxidation of cyclohexane by hydroperoxide to produce KA oil. Heterogeneous catalysts processes have the advantage of easy separation and have been reported to catalyze the oxidation of cyclohexane by hydroperoxide. Many heterogeneous catalysts are based on zeolite-like supports in which transition metals or noble metals are incorporated or implemented, or on oxide supports on which transition metals are deposited.

GB 964,869 discloses a process for the oxidation of liquid cyclohexane to cyclohexanol and cyclohexanone by means of free oxygen, wherein in the course of the oxidation the reaction mixture is subjected to reduction whereby cyclohexanone and cyclohexyl hydroperoxide are converted into cyclohexanol. The reduction can be carried out by catalytic hydrogenation or by means of chemical (non-catalytic) reducing agents. As hydrogenation catalysts, there are mentioned catalysts based upon nickel, copper, platinum, palladium, ruthenium and rhodium. The catalysts are preferably deposited upon a solid support disposed in a fixed bed, over which the material to be hydrogenated trickles in counter-current to the hydrogen. As chemical reducing agents, there may be employed metals which, on contact with the acids formed, liberate nascent hydrogen, or hydrides such as alkali borohydrides or lithiumaluminium hydride.

U.S. Pat. No. 3,479,394 discloses a process for the preparation of cyclohexanol and cyclohexanone, by air oxidation of cyclohexane and stopping the oxidation when a relatively low proportion of hydroperoxide has been formed, and thereafter converting the hydroperoxide into cyclohexanol and cyclohexanone. This conversion may be effected by chemical reduction either with hydrogen in the presence of catalysts, e.g. platinum or Raney nickel, or with salts of metals wherein the metal is in its lowest valance state, e.g. ferrous sulphate.

Gerd Dahlhoff et al.: “ε-Caprolactam: new by-product free synthesis routes”, Catalysis Reviews: Science and Engineering, vol. 43, no. 4, pages 381-441 discloses that ε-caprolactam can be produced from cyclohexanone via cyclohexanone oxime.

U.S. Pat. No. 3,772,375 A discloses hydrogenation of 6-hydroxyperoxyhexanoic acid isolated from an aqueous wash of the product of oxidising cyclohexane with molecular oxygen in the liquid phase. The 6-hydroxyperoxyhexanoic acid is subjected to hydrogenation as such or as salt contained in the aqueous phase in the presence of a catalyst consisting essentially of metallic palladium, rhodium or platinum.

U.S. Pat. No. 3,937,735 discloses a process for the preparation of cyclohexanone which comprises oxidizing cyclohexane in the liquid phase with oxygen or an oxygen-containing gas, to produce an oxidation reaction product containing cyclohexyl hydroperoxide, catalytically hydrogenating the oxidation product in a hydrogenation zone in the presence of a catalyst containing palladium, platinum, nickel or rhodium with a hydrogen gas-containing stream, whereby the cyclohexyl hydroperoxide is converted substantially to cyclohexanol, recovering the cyclohexanol fraction by distillation and catalytically dehydrogenating the cyclohexanol, to cyclohexanone and hydrogen, separating the said cyclohexanone and passing the resulting hydrogen gas-containing stream to the said hydrogenating zone to effect the said hydrogenation of the oxidation product. The catalyst is preferably deposited on a carrier, e.g. aluminium oxide, carbon or silica. In the examples a supported palladium catalyst in a fixed bed containing 0.1% by weight palladium on aluminium oxide is used.

There remains a need for a process for the oxidation of cyclohexane into a product mixture containing cyclohexanone and cyclohexanol with high conversion of cyclohexane and high selectivity to KA oil with low cost of catalyst preparation. The object of the present invention is to provide such a process.

### SUMMARY OF THE INVENTION

The object is solved by a method for preparing a mixture containing cyclohexanol and cyclohexanone, comprising the step of hydrogenating cyclohexyl hydroperoxide in cyclohexane in the presence of a Raney nickel catalyst to give cyclohexanol and cyclohexanone.

Preferably, the method comprises the steps
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone.

In one embodiment of the invention, step b) is carried out in the reaction mixture obtained in step a).

In another embodiment of the invention, prior to step b), the reaction mixture obtained in step a) is extracted with water to give an organic phase containing cyclohexyl hydroperoxide, cyclohexanol, cyclohexanone and unconverted cyclohexane and an aqueous phase containing 6-hydroxyperoxycaproic acid, and step b) is carried out in the organic phase.

In further embodiments of the invention, 6-hydroxyperoxycaproic acid is hydrogenated in the presence of a Raney nickel catalyst to give 6-hydroxycaproic acid.

In a preferred embodiment, 6-hydroxyperoxycaproic acid is hydrogenated in the aqueous phase in the presence of a Raney nickel catalyst to give 6-hydroxycaproic acid.

The present invention also relates to a method for preparing adipic acid, comprising the steps of
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone, and
c) oxidizing cyclohexanol and cyclohexanone, optionally after purification by distillation,with nitric acid to give adipic acid.

The invention further concerns a method for preparing 6-hydroxycaproic acid, comprising the step of hydrogenating 6-hydroxyperoxycaproic acid in the presence of a Raney nickel catalyst.

Preferably, the method comprises the steps of
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane, and
b1) hydrogenating 6-hydroxyperoxycaproic acid in the presence of a Raney nickel catalyst to give 6-hydroxycaproic acid.

In one embodiment, step b1) is carried out in the reaction mixture obtained in step a).

In a further embodiment, prior to step b1), the reaction mixture obtained in step a) is extracted with water to give an organic phase containing cyclohexyl hydroperoxide, cyclohexanol, cyclohexanone and unconverted cyclohexane and an aqueous phase containing 6-hydroxyperoxycaproic acid, and step b1) is carried out in the aqueous phase.

### DETAILED DESCRIPTION

Generally, in a first step a), cyclohexane is oxidized with molecular oxygen to give a reaction mixture comprising cyclohexyl hydroperoxide, cyclohexanol, cyclohexanone, 6-hydroxyperoxycaproic acid, unconverted cyclohexane and possibly further by-products.

Step a) can be carried out by thermal auto-oxidation of cyclohexane under pressure, e.g. at 15-25 bar, and at high temperature, e.g. at 160-190° C., with molecular oxygen, preferably in admixture with an inert gas.

In step b), cyclohexyl hydroperoxide is hydrogenated in the presence of a Raney nickel catalyst to give cyclohexanol and cyclohexanone.

In step b1) 6-hydroxyperoxycaproic acid can be hydrogenated in the presence of a Raney nickel catalyst to give 6-hydroxycaproic acid. 6-hydroxyperoxycaproic acid can be hydrogenated concurrently with cyclohexyl hydroperoxide in the same reaction mixture, or hydroxyperoxycaproic acid can be separated from cyclohexyl hydroperoxide before hydrogenation and hydrogenated in a separate step b1).

Suitable Raney catalysts can have, for example, a BET surface from 80 to 120 m²/g and can contain promotor elements, such as zinc or chromium.

The Raney catalyst used according to the present invention can be prepared in the usual manner. A Ni—Al alloy is prepared by dissolving nickel in molten aluminium followed by cooling (“quenching”). Small amounts of a third metal, such as zinc or chromium or others, can be added as promotor to enhance the activity of the resulting catalyst. The promoter changes the mixture from a binary alloy to a ternary alloy, which can lead to different quenching and leaching properties during activation.

In the activation process, the alloy, usually as a fine powder, is treated with a concentrated solution of sodium hydroxide. The formation of sodium aluminate (Na[Al(OH)4]) requires that solutions of high concentration of sodium hydroxide. Sodium hydroxide solutions with concentrations of up to 5 M are commonly used. Commonly, leaching is conducted between 70 and 110° C.

In the practice of the invention, the catalyst can be slurried with reaction mixtures using techniques known in the art. The process of the invention is suitable for either batch, semi-continuous or continuous cyclohexyl hydroperoxide hydrogenation. These processes can be performed under a wide variety of conditions, as will be apparent to persons of ordinary skill.

Suitable reaction temperatures for the process of the invention typically range from about 20 to about 80° C. or higher, advantageously from about 25 to about 60° C. ° C.

The process according to the invention is performed advantageously at a hydrogen pressure from 0.1 MPa (1 bar) to 10 MPa (100 bar), preferably from 0.1 MPa (1 bar) to 5 MPa (50 bar), e.g. at 2 MPa (20 bar).

At the end of the hydrogenation reaction, the compound of interest may be eventually purified by well-known methods of the technical field, such as distillation.

In a further step c), cyclohexanol and cyclohexanone can be oxidized with nitric acid to give adipic acid.

Step c) can be carried out by nitric acid oxidation of KA oil in concentrated nitric acid at atmospheric pressure or under elevated pressure. The reaction temperature is between 70 and 100° C. Homogeneous transitions metals can catalyze the reaction. Adipic acid and by-products can be purified by series crystallization.

In further steps, cyclohexanol can be dehydrogenated to give further cyclohexanone, and cyclohexanone can be converted to epsilon-caprolactam.

Thus, the invention also concerns a method for preparing epsilon-caprolactam, comprising the steps
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone,
c) optionally purifying cyclohexanol and cyclohexane by distillation,
d) optionally separating cyclohexanone from cyclohexanol,
e) dehydrogenating cyclohexanol to cyclohexanone,
f) converting cyclohexanone to epsilon-caprolactam.

Preferably, in further steps, cyclohexanol can be dehydrogenated to give further cyclohexanone, and cyclohexanone can be reacted with hydroxylamine to give, via cyclohexanonoxim, epsilon-caprolactam. The present invention thus also concerns a method for preparing epsilon-caprolactam, comprising the steps
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone,
c) optionally purifying cyclohexanol and cyclohexanone by distillation,
d) optionally separating cyclohexanone from cyclohexanol,
e) dehydrogenating cyclohexanol to cyclohexanone,
f1) reacting cyclohexanone with hydroxylamine or its salt to give cyclohexanonoxim,

2) reacting cyclohexanonoxim to give epsilon-caprolactam.

Step d) is optional. The purified KA oil cantaining cyclohexanol and cyclohexanone can be subject to dehydrogenation without separation of cyclohexanone and cyclohexanol.

Step e) can be done at, for example, at 200 to 450° C., preferably about 270° C., in the presence of a zinc or copper containing dehydrogenation catalyst.

Step f) is commonly carried out with aqueous hydroxylamine sulfate or with a hydroxylamine and phosphoric acid containing buffer solution.

Step g) (Beckmann-rearrangement) is commonly carried out in the presence of concentrated sulfuric acid or oleum, at a temperature of preferably from 90 to 120° C. The formed lactam sulfate-solution is usually neutralized with ammonia to give the free lactam.

Further methods for converting cyclohexanone to epsilon-caprolactam can be found in the literature.

The present invention is further illustrated by the following examples. It should be understood that the following examples are for illustration purposes only, and are not used to limit the present invention thereto.

### EXAMPLES

### Analyses

Yields and selectivity was determined using gas chromatography with an internal standard. CyOOH in cyclohexane was quantified by iodometry.

Conversion=conversion of CyOOH. In the case of CyOOH decomposition, conversion is defined as the number of moles of CyOOH consumed divided by the initial number of moles of CyOOH:

Conversion=100×nCyOOH(consumed)/nCyOOH(initial)

In the case of CyOOH decomposition, selectivity is defined as the number of moles of cyclohexanol (CyOH) and cyclohexanone (CyO) produced divided by the number of moles of CyOOH consumed:

100×(nCyOH(produced)+nCyO(produced))/nCyOOH(consumed)

Yield=Conversion×Selectivity

### Raw Materials

Industrial Reaction Mixture used in the Examples:

1. Reaction mixture A, mixture of cyclohexylhydroperoxide (CyOOH) and 6-hydroxyperoxycaproic acid (HPOCap): cyclohexane is oxidized with molecular oxygen or mixtures of molecular oxygen and other gases which are inert to give a reaction mixture which comprises, as main components, CyOOH, cyclohexanol (CyOH), cyclohexanone (CyO), unconverted cyclohexane, HPOCap and other carboxylic and dicarboxilic acids having from 1 to 6 carbons.

The reaction mixture A, after adding water in a washing column, is separated into an organic phase (reaction mixture B) and an aqueous phase (reaction mixture C).

2. Reaction mixture B, CyOOH: After washing reaction mixture A with water, the organic phase is mainly composed of cyclohexane, cyclohexanone, cyclohexanol, CyOOH and other carboxylic and dicarboxilic acids having from 1 to 6 carbons.

3.

4. Reaction mixture C, HPOCap: After washing reaction mixture A with water, the aqueous phase is mainly composed of HPOCap and other carboxylic and dicarboxilic acids having from 1 to 6 carbons.

### Example 1: Conversion of Reaction Mixture B using the Current Industrial Chromium Based Catalyst

A reference experiment was conducted batchwise with the current industrial catalyst based on chromium, used for the conversion of reaction mixture B to KA oil. 42.7 g of reaction mixture B, containing approximatively 6% of cyclohexylhydroperoxide in cyclohexane, were poured in a glass reactor equipped with a Dean Stark filled with cyclohexane. The temperature was raised at 80° C. and 0.1 g solution containing 0.5% of chromium catalyst was added to the reaction mixture B. The results obtained are reported in the following table.

Conversion=conversion of CyOOH. In the case of CyOOH decomposition, conversion is defined as the number of moles of CyOOH consumed divided by the initial number of moles of CyOOH:

Conversion=100×nCyOOH(consumed)/nCyOOH(initial)

In the case of CyOOH decomposition, selectivity is defined as the number of moles of cyclohexanol (CyOH) and cyclohexanone (CyO) produced divided by the number of moles of CyOOH consumed:

100×(nCyOH(produced)+nCyO(produced))/nCyOOH(consumed)

Yield=Conversion×Selectivity

| Conversion | Yield (%) | | | |
|---|---|---|---|---|
| (%) | Total | Cyclohexanol | Cyclohexanone | By-products |
| 99 | 98.8 | 20.5 | 78.3 | 1.7 |

Molar percentages of the main by-products in the crude reaction mixture are reported below:

| | % |
|---|---|
| Propionic acid | 0.56 |
| Valeric acid | 0.43 |
| Caproic acid | 0.18 |
| 1,2-t-cyclohexanediol | 0.09 |
| 6-hydroxycaproic acid | 0.28 |
| peroxydicyclohexane | 0.32 |
| Unknown | 2.02 |

### Example 2: General Procedure for Reaction Mixture B Batchwise Hydrogenation over Nickel Raney Catalyst

In a dry atmosphere of N₂, 0.3 g of nickel Raney catalyst were stirred in the hydrogenation autoclave with 68 g of reaction mixture B, containing approximately 6% of cyclohexylhydroperoxide in cyclohexane. The temperature was raised at 60° C. and 20 bar of hydrogen overall pressure. After 2 hours, the crude reaction mixture produced was analyzed by gas chromatography. The results obtained are reported in the following table.

| Conversion | Yield (%) | | | |
|---|---|---|---|---|
| (%) | Total | Cyclohexanol | Cyclohexanone | By-products |
| 100 | 102 | 86.9 | 15.3 | −0.5 |

Since the starting reaction mixture B already contains impurities, the hydrogenation of reaction mixture B leads to a decrease of those impurities in the reaction medium. Thus, the amount of impurities is lower after hydrogenation than before.

Molar percentages of the main by-products in the crude reaction mixture are reported below:

| | % |
|---|---|
| Propionic acid | 0.58 |
| Valeric acid | 0.36 |
| Caproic acid | 0.17 |
| 1,2-t-cyclohexanediol | 0.26 |
| 6-hydroxycaproic acid | 0.13 |
| peroxydicyclohexane | 0.12 |
| Unknown | 1.24 |

The overall performances of reaction mixture B conversion into KA oil were improved with batchwise hydrogenation over nickel Raney catalyst compared to those obtained with chromium catalyst. The cyclohexylhydroperoxide transformation rate (or conversion) and KA oil yield are higher and by-products formation is lower than those obtained with chromium catalyst. The yield in by-products is negative because the initial reaction mixture B already contained by-products before hydrogenation reaction. Cyclohexanol is the main product of CyOOH hydrogenation.

### Example 3: General Procedure for Semi-Continuous Reaction Mixture B Hydrogenation over Nickel Raney Catalyst

In a dry atmosphere of N₂, 0.1 g of nickel Raney catalyst were stirred in the hydrogenation autoclave with 5.6 g of cyclohexane. The temperature was raised at 60° C. and 20 bar of hydrogen overall pressure. 19 g of reaction mixture B were added dropwise at a mass flow of 15 g/h and were hydrogenated. After 1.5 hours, the crude reaction mixture produced was analyzed by gas chromatography. The results obtained are reported in the following table.

| Conversion | Yield (%) | | | |
|---|---|---|---|---|
| (%) | Total | Cyclohexanol | Cyclohexanone | By-products |
| 99.6 | 95.0 | 80.8 | 14.2 | −1.9 |

Molar percentages of the main by-products in the crude reaction mixture are reported below:

| | % |
|---|---|
| Propionic acid | 0.54 |
| Valeric acid | 0.17 |
| Caproic acid | 0.12 |
| 1,2-t-cyclohexanediol | 0.27 |
| 6-hydroxycaproic acid | 0.02 |
| peroxydicyclohexane | 0.10 |
| Unknown | 0.92 |

The by-products yield is lower in semi continuous hydrogenation than that obtained batchwise.

### Example 4: Effect of Temperature

In a dry atmosphere of N₂, 0.054 g of nickel Raney catalyst were stirred in the autoclave with 5.6 g of cyclohexane. The temperature was raised at the set point value and 20 bar of hydrogen overall pressure. 12.32 g of reaction mixture B were added in one time and were hydrogenated. The crude reaction mixture produced was analyzed by gas chromatography. The results obtained are reported in the following table.

| | | Yield (%) | | | |
|---|---|---|---|---|---|
| | Conversion | | | | By- |
| T (° C.) | (%) | Total | Cyclohexanol | Cyclohexanone | products |
| 25 | 98 | 97.7 | 83.1 | 14.6 | −0.84 |
| 45 | 100 | 104.0 | 94.0 | 10.0 | — |
| 60 | 100 | 104.3 | 99.8 | 4.5 | −0.50 |

Molar percentages of the main by-products in the crude reaction mixture are reported below:

| | 25 | 45 | 60 |
|---|---|---|---|
| Temperature (° C.) | % | | |
| Propionic acid | 0.39 | 0.40 | 0.40 |
| Valeric acid | 0.24 | 0.25 | 0.25 |
| Caproic acid | 0.10 | 0.11 | 0.12 |
| 1,2-t-cyclohexanediol | 0.14 | 0.18 | 0.19 |
| 6-hydroxycaproic acid | 0.07 | 0.17 | 0.17 |
| peroxydicyclohexane | 0.00 | 0.00 | 0.00 |

The catalytic activity was measured at each reaction temperature:

| | Catalytic activity |
|---|---|
| T (° C.) | (10⁻⁵ mol H₂/g/s) |
| 25 | 6.9 |
| 45 | 7.7 |
| 60 | 8.6 |

More cyclohexanone was obtained at lower temperature meaning that hydrogenation of cyclohexanone to cyclohexanol is the main side reaction.

### Example 5: Re-Use of the Catalyst

The procedure of example 2 was followed. Then the recovered nickel Raney catalyst was added again to the system and the hydrogenation was carried out cyclically at 60° C. The results obtained are reported in the following table.

| | Conversion | Yield KA oil |
|---|---|---|
| Cycle No. of the catalyst | (%) | (%) |
| Fresh catalyst | 100 | 99.9 |
| 1 | 100 | 99.6 |
| 2 | 100 | 99.9 |

### Example 6: Hydrogenation of Reaction Mixture C

The procedure of example 2 was followed except that the reaction mixture C was hydrogenated. In a dry atmosphere of N₂, 0.43 g of nickel Raney catalyst were stirred with 26 g of reaction mixture C, containing approximately 10% of 6-hydroxyperoxycaproic acid (HPOCap). The temperature was raised at 60° C. and 20 bar of hydrogen overall pressure. After 1 hour, the transformation rate of HPOCap was 100%.

### Example 7: Hydrogenation of Reaction Mixture A

The procedure of example 4 was followed except that the reaction mixture A was hydrogenated. In a dry atmosphere of N₂, 0.061 g of nickel Raney catalyst were stirred with 5.7 g of cyclohexane. The temperature was raised at 60° C. and 20 bar of hydrogen overall pressure. 12.7 g of reaction mixture A, containing approximately 6.5% of hydroperoxides (CyOOH+HPOCap) were added in one time in the autoclave and were hydrogenated. The crude reaction mixture produced was analyzed by gas chromatography. The results obtained are reported in the following table.

| | | Yield (%) | | | |
|---|---|---|---|---|---|
| TT_{HPOCap*} | Conversion | | | | By- |
| (%) | (%) | Total | Cyclohexanol | Cyclohexanone | products |
| 85.1 | 85.3 | 80.0 | 54.5 | 25.5 | −0.2 |

*TT_{HPOCap} = Conversion of HPOCap

1.-12. (canceled)

13. A method for preparing a mixture containing cyclohexanol and cyclohexanone, comprising the step of hydrogenating cyclohexyl hydroperoxide in cyclohexane in the presence of a Raney nickel catalyst to give cyclohexanol and cyclohexanone, comprising the steps of
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone,
wherein, prior to step b), the reaction mixture obtained in step a) is extracted with water to give an organic phase containing cyclohexyl hydroperoxide, cyclohexanol, cyclohexanone and unconverted cyclohexane and an aqueous phase containing 6-hydroxyperoxycaproic acid, and step b) is carried out in the organic phase.

14. The method according to claim 13, wherein 6-hydroxyperoxycaproic acid is hydrogenated in the presence of a Raney nickel catalyst to give 6-hydroxycaproic acid.

15. The method according to claim 13, wherein 6-hydroxyperoxycaproic acid is hydrogenated in the aqueous phase in the presence of a Raney nickel catalyst to give 6-hydroxycaproic acid.

16. A method for preparing adipic acid, comprising the steps
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone,
c) oxidizing cyclohexanol and cyclohexanone with nitirc acid to give adipic acid,
wherein, prior to step b), the reaction mixture obtained in step a) is extracted with water to give an organic phase containing cyclohexyl hydroperoxide, cyclohexanol, cyclohexanone and unconverted cyclohexane and an aqueous phase containing 6-hydroxy-peroxycaproic acid, and step b) is carried out in the organic phase.

17. The method according to claim 16, wherein 6-hydroxyperoxycaproic acid is hydrogenated in the presence of a Raney nickel catalyst to give 6-hydroxycaproic acid.

18. The method according to claim 16, wherein 6-hydroxyperoxycaproic acid is hydrogenated in the aqueous phase in the presence of a Raney nickel catalyst to give 6-hydroxycaproic acid.

19. A method for preparing 6-hydroxycaproic acid, comprising the step of hydrogenating 6-hydroxyperoxycaproic acid in the presence of a Raney nickel catalyst.

20. The method according to claim 19, comprising the steps of
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating 6-hydroxyperoxycaproic acid in the presence of a Raney nickel catalyst to give 6-hydroxycaproic acid.

21. The method according to claim 20, wherein step b1) is carried out in the reaction mixture obtained in step a).

22. The method according to claim 20, wherein, prior to step b1), the reaction mixture obtained in step a) is extracted with water to give an organic phase containing cyclohexyl hydroperoxide, cyclohexane, cyclohexanone and unconverted cyclohexane and an aqueous phase containing 6-hydroxyperoxy-caproic acid, and step b) is carried out in the aqueous phase.

23. A method for preparing epsilon-caprolactam, comprising the steps
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone,
c) optionally purifying cyclohexanol and cyclohexane by distillation,
d) optionally separating cyclohexanone from cyclohexanol,
e) dehydrogenating cyclohexanol to cyclohexanone,
f) converting cyclohexanone to epsilon-caprolactam,
wherein, prior to step b), the reaction mixture obtained in step a) is extracted with water to give an organic phase containing cyclohexyl hydroperoxide, cyclohexanol, cyclohexanone and unconverted cyclohexane and an aqueous phase containing 6-hydroxy-peroxycaproic acid, and step b) is carried out in the organic phase.

24. The method according to claim 23 for preparing epsilon-caprolactam, comprising the steps
a) oxidizing cyclohexane with molecular oxygen to give a reaction mixture comprising cyclohexyl hydroperoxide, cyclohexanol, cyclohexanone, 6-hydroxyperoxycaproic acid and unconverted cyclohexane,
b) hydrogenating cyclohexyl hydroperoxide in the presence of a Raney nickel catalyst to give cyclohexanol and cyclohexanone,
c) optionally purifying cyclohexanol and cyclohexanone by distillation,
d) optionally separating cyclohexanone from cyclohexanol,
e) dehydrogenating cyclohexanol to cyclohexanone,
f1) reacting cyclohexanone with hydroxylamine or its salt to give cyclohexanonoxim,
f2) reacting cyclohexanonoxim to give epsilon-caprolactam,
wherein, prior to step b), the reaction mixture obtained in step a) is extracted with water to give an organic phase containing cyclohexyl hydroperoxide, cyclohexanol, cyclohexanone and unconverted cyclohexane and an aqueous phase containing 6-hydroxy-peroxycaproic acid, and step b) is carried out in the organic phase.
